(19) United States
(12) Patent Application Publication
(10) Pub. No.: US 2012/0059388 A1
(43) Pub. Date: Mar. 8, 2012

(54) Method of placing a surgical mesh for a laparoscopic surgical procedure and an insertion tube to facilitate surgical mesh placement

(76) Inventor: Robert C. Knowles, Norfolk, VA (US)

(21) Appl. No.: 12/807,465

(22) Filed: Sep. 7, 2010

(51) Int. Cl. A61B 17/00

(52) U.S. Cl. 606/108; 606/151

(57) ABSTRACT

A method of laparoscopically introducing a piece of surgical mesh into the abdominal cavity of a patient through a small incision. The mesh is tightly rolled after having traction sutures placed therein and being hydrated. The surgical mesh is pulled into a prelubricated insertion tube having been ligated adjacent its proximal end. A grasper is used to pull mesh into the tube. The tube/mesh is inserted into the abdominal cavity through a small incision. Once inside the abdomen, the insertion tube is split to release the surgical mesh that may then be used in the surgery. Also provided is an insertion tube having a thinned or otherwise weakened region extending along a longitudinal axis thereof. A ripcord extending along the insertion tube from its ligated, proximal end to beyond the distal end is utilized to spilt the insertion tube to facilitate freeing the surgical mesh from within.

### FIELD OF THE INVENTION

The invention pertains to a method of placing a surgical mesh material into a body cavity laparoscopically, and, more particularly, to a method of introducing relatively large surgical meshes through small incisions, for example, the incision made by a 5 mm trocar. In addition, an insertion is provided to facilitate the surgical mesh placements.

### BACKGROUND OF THE INVENTION

Laparoscopic surgical techniques are both well known and widely practiced for performing a wide variety of surgical procedures. The major advantage of laparoscopic procedures is that no large incision need be made into a patient, thereby greatly reducing patient recovery time. In some cases, simple procedures performed laparoscopically may be done either on an outpatient basis, or with a limited hospitalization. Such procedures once may have required a multi-day hospitalization when conventional surgical techniques were used.

Laparoscopic surgery typically utilizes multiple trocars for the insertion of a camera and surgical instruments, and the introduction of materials such as sutures, repair meshes, and the like required for the specific surgical procedure. One or more additional trocars may be used to inflate the abdomen or other body cavity to facilitate the surgery being performed. The camera provides an image on a monitor which is used by the surgeon to guide his or her manipulation of the instruments.

It has been observed that patient discomfort is proportional to the diameter of the trocars utilized for the surgery, large diameter trocars resulting in more discomfort, small diameter trocars resulting in less discomfort. It has also been recognized that a puncture or incision made by a small diameter, for example, a 5 mm trocar may be virtually self healing requiring no suture to close the puncture or incision (i.e., fascial defect) upon withdrawal of the trocar. This provides additional incentive to utilize small diameter trocars whenever possible.

One typical class of surgery commonly performed laparoscopically is the repair of hernias. A support mesh is typically utilized for such hernia repair. The mesh must, of course, be introduced into the abdominal cavity through a trocar.

One specific type of hernia frequently repaired laparoscopically is a ventral hernia. Such hernias often occur in the abdominal wall at a site where a surgical incision was previously made. At such a site the abdominal muscles may have weakened resulting in a bulge or a tear. This situation may be likened to the way that an inner tube pushes through a damaged tire. The inner lining of the abdomen may push through the weakened area of the abdominal wall to form a balloon-like sac. This can allow a loop of intestines or other abdominal contents to push into the sac. If the abdominal contents get stuck within the sac, they can become trapped or “incarcerated.” This incarceration may lead to potentially serious problems that might conceivably require emergency surgery.

To repair a ventral hernia, a patch of surgical mesh is inserted into the abdomen and used to reinforce the abdominal wall. In this approach, a laparoscope (a tiny telescope with a television camera attached) is inserted through a cannula (e.g., a trocar). The laparoscope and TV camera allow the surgeon to view the hernia from the inside. Other small incisions will be required for other small cannulas for placement of other instruments to remove any scar tissue and to insert a surgical mesh into the abdomen. This mesh, or screen, is fixed under the hernia defect to the strong tissues of the abdominal wall. It is typically held in place with special surgical tacks and in many instances, sutures. Usually, three or four ¼ inch to ½ inch incisions are necessary. The sutures, which go through the entire thickness of the abdominal wall, are placed through smaller incisions around the circumference of the mesh. This surgery is usually performed under general anesthesia.

The size of the mesh required for the repair of ventral hernias is typically relatively large, for example 15×20 cm and 20×25 cm are typical mesh sizes. Procedures of the prior art require large trocars to effect the insertion thereof.

### DISCUSSION OF THE RELATED ART

Apparatus for the insertion of mesh into a body cavity may be found in the prior art. For example, Published United States Patent Application No. 2004/0092970 for PROSTHETIC MESH ANCHOR DEVICE, published May 13, 2004 upon application by Alfredo F. Xavier teaches a device wherein percutaneous mesh may be introduced into a patient's abdominal cavity, the mesh no larger than 11×14 cm. The diameter of the Xavier apparatus is relatively large (i.e., 10 mm) trocar. The apparatus is intended for mesh placement for the repair of hernias in the groin region.

This published patent application is not seen to teach or suggest the novel apparatus for and method of laparoscopically introducing a relatively large mesh for the repair of a ventral hernia in accordance with the present invention.

### SUMMARY OF THE INVENTION

In accordance with the present invention there is provided a method of laparoscopically introducing relatively large pieces of surgical mesh into the abdominal cavity of a patient using a small incision, typically the incision made by a 5 mm diameter trocar. After placing traction sutures (typically at least four) around the perimeter of the mesh adjacent the outside edges, the surgical mesh is hydrated and tightly rolled.

An insertion tube is ligated adjacent a proximal end and a small nick is cut through the tube wall adjacent the ligation. After lubricating the lumen of the insertion tube with surgical jelly, a laparoscopic grasper, for example, a 5 mm Maryland dissector is inserted into the lumen through the small nick. The grasper is advanced until the grasping elements protrude from the insertion tube at the distal end thereof.

The grasper is used to pull the hydrated, tightly rolled surgical mesh into the lubricated lumen of the insertion tube.

The proximal, ligated end of the insertion tube bearing the tightly rolled surgical mesh is inserted into the patient's abdominal cavity through a very small incision, typically an incision formed by a 5 mm trocar.

Once inserted, the insertion tube is split or otherwise opened to release the surgical mesh that may then be used to perform the necessary surgery.

A novel insertion tube having a thinned wall region extending along a longitudinal axis and a ripcord extending from a ligated, proximal end back to a point beyond a distal end of the insertion tube may be utilized to facilitate splitting the insertion tube to facilitate release of the rolled surgical mesh from within the insertion tube.

The surgical mesh is typically utilized to laparoscopically repair a ventral hernia.

It is, therefore, an object of the invention to provide a method of introducing a relatively large surgical repair mesh into the abdominal cavity through a small diameter incision (e.g., an incision made by 5 mm trocar).

It is an additional object of the invention to provide an insertion tube to facilitate insertion of surgical mesh through such small incisions.

It is a further object of the invention to provide an insertion tube weakened area along a longitudinal axis thereof to facilitate opening of the insertion tube.

It is a still further object of the invention to provide an insertion tube that may readily be parted or separated from outside the abdominal cavity of a patient using a ripcord to part the insertion tube along the weakened area thereof.

### BRIEF DESCRIPTION OF THE DRAWINGS

Various objects, features, and attendant advantages of the present invention will become more fully appreciated as the same becomes better understood when considered in conjunction with the accompanying drawings, in which like reference characters designate the same or similar parts throughout the several views, and wherein:

FIG. 1a is a pictorial, schematic view of some of the materials and instruments utilized to practice the method of the invention;

FIG. 1b is a pictorial, schematic view of the instruments of FIG. 1a and wherein an insertion tube is ligated at a proximal end thereof;

FIG. 2 is a top, perspective, schematic view of a piece of surgical mesh prepared by placing traction sutures around the perimeter thereof;

FIG. 3 is a perspective of the ligated insertion tube of FIG. 1b with a laparoscopic grasper inserted therethrough;

FIG. 4 is a side, perspective view of the ligated insertion tube of FIG. 1b with rolled surgical mesh partially inserted in the lumen thereof;

FIG. 5a is a side, perspective of an insertion tube in accordance with the invention;

FIG. 5b is an end view of the insertion tube of FIG. 5a; and

FIG. 5c is a top plan view of the insertion tube of FIGS. 5a and 5b.

### DETAILED DESCRIPTION OF THE PREFERRED EMBODIMENT

The present invention provides an apparatus for and method of inserting a large piece of surgical mesh into the abdominal cavity of a patient through a small incision, typically the incision formed by a 5 mm diameter trocar. As used herein, the term trocar is used to apply to any suitable small diameter cannula that may be used to provide access to a body cavity of a patient.

As previously stated, the use of small diameter trocars is highly desirable for several reasons. First, small diameter trocars minimize patient discomfort. Also fascial defects resulting from the use of a 5 mm trocar usually do not require a suture for closing (i.e., they are generally self sealing) whereas fascial defects from larger diameter trocars generally do require suturing. Finally, the use of small diameter trocars minimizes the risk of future herniation at the site.

The surgical mesh typically used to repair ventral hernias is typically a bilaminate having a first major surface that is bioresorbable while an opposing major surface is formed from non-bioresorbable material. One commercially available product Proceed™ Surgical Mesh manufactured by Ethicon, Inc., a division of Johnson and Johnson. Proceed™ Surgical Mesh is composed of an oxidized regenerated cellulose (ORC) fabric and a nonabsorbable polypropylene mesh that is encapsulated by a polydioxanone polymer. The polypropylene side of the mesh allows for tissue ingrowth while the ORC side of the mesh is a bioresorbable layer that separates the polypropylene mesh from the underlying tissue during the healing process to minimize tissue attachment thereto. There are other products, for example Seprafilm®, well known to those of skill in the art that provide similar bilaminate structure having both a tissue ingrowth side and an opposing side that is bioresorbable.

Another mesh product suitable for practicing the method of the present invention is Parietex™ dual-sided mesh composite provided by Covidien whose US headquarters is in Mansfield, Mass. Parietex™ is a polyester mesh/resorbable film that is claimed to provide optimal tissue in-growth and fewer visceral attachments. A protective collagen-based barrier is provided to help prevent tissue attachment. Parietex™ features increased rigidity during implantation allowing ease of handling. However, the polyester material softens and conforms to the anatomy once implanted.

Surgical mesh forms no part of the present invention, it will be recognized that any suitable surgical mesh having appropriate properties for the particular surgery being undertaken may be used. While Proceed™ or Parietex™ surgical mesh is used for purposes of disclosure, they are not considered limiting.

For ventral hernia repair, surgical mesh in 15×25 cm and 20×25 cm is commonly used. It will be recognized that other sizes of surgical mesh may be required for a particular ventral hernia repair or other similar surgery. Consequently, the invention is not considered limited to a particular size surgical mesh. Rather, the invention includes any and all surgical mesh sizes.

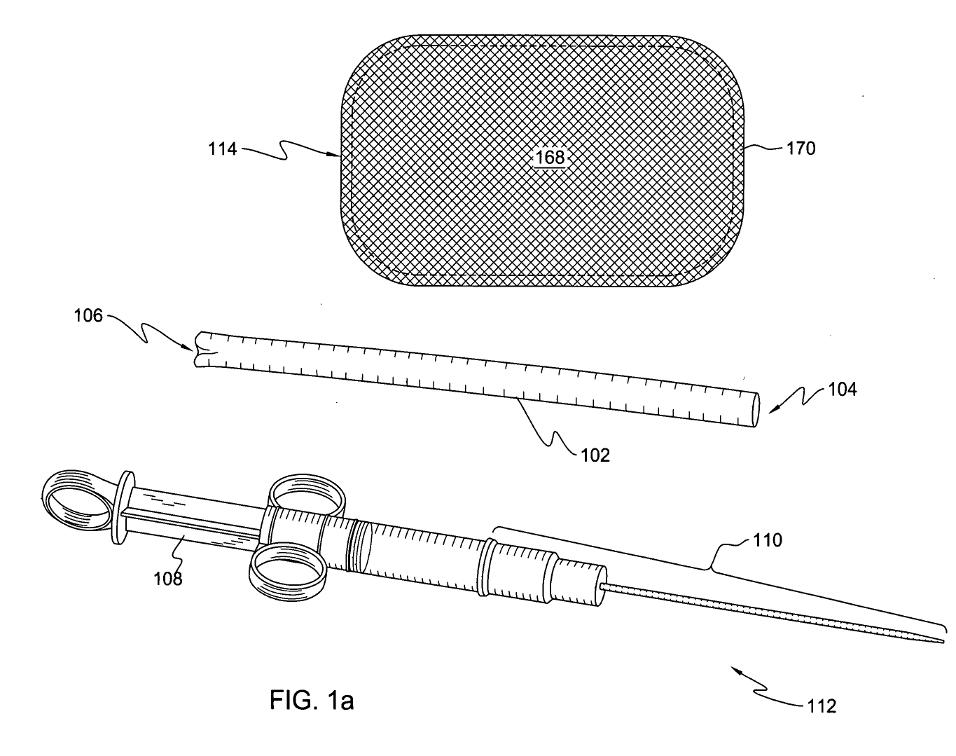

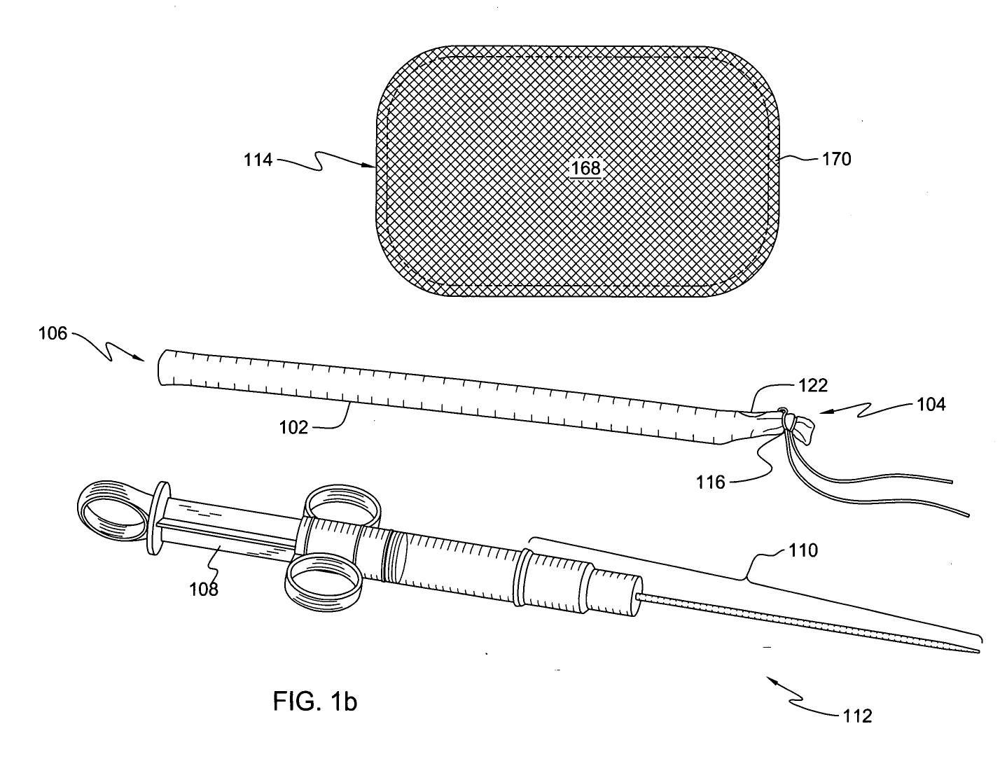

Referring first to FIG. 1a, there is shown a pictorial representation of several components required to practice the method of the present invention.

An elongated, hollow, elastic insertion tube having a diameter in the range of approximately 9-10 mm 102 has an open proximal end 104 and an open distal end 106. Insertion tube 102 is typically a silicon rubber or similar elastic material that may readily be dilated. Suitable materials are believed to be known to those of skill in the art.

A syringe 108 of approximately ______ cc capacity [Dr. Knowles?] is filled with surgical jelly, not specifically identified. Surgical jelly is believed to be well known to those of skill in the art and is not further described or discussed herein. Syringe 108 has a flexible, tapered tip 110 attached to its discharge end 112. Tapered tip 110 is sized and configured to fit into distal end 106 of insertion tube 102 and is similar to a 20 gauge “angiocath” tip. Angio catheters are believed to be well known to those of skill in the art and flexible, tapered tip 110 is not further discussed herein.

A piece of surgical mesh 114 is shown proximate insertion tube 102. Surgical mesh 114 is selected from one of the exemplary bi-layer surgical meshes described in detail hereinabove. Surgical mesh 114 as seen in FIG. 1 has a central region 168 with a perimeter region 170 where the absorbable layer is partially visible.

In the method of the invention, first the proximal end 104 of insertion tube 102 is suture ligated at a proximal end 104 thereof as seen in FIG. 1b. A traction suture 116, such as an Ethibond® suture supplied by Ethicon, Inc. of Bridgewater, N.J. has been found suitable for the application. Ethibond® sutures are nonabsorbable, braided, sterile, surgical sutures typically composed of Poly (ethylene, terephthalate). The sutures are prepared from fibers of high molecular weight, long-chain, linear polyesters having recurrent aromatic rings as an integral component. They are uniformly coated with polybutilate or poly {oxy-1,4 butanediyloxy (1,6-dioxo-1,6 hexanediyl)}. The highly adherent coating is a relatively nonreactive nonabsorbable compound which acts as a lubricant to mechanically improve the physical properties of the uncoated sutures by improving handling quality as compared to the braided, uncoated fiber. The sutures are braided for optimal handling properties, and typically dyed green for enhanced visibility in the surgical field. It will be recognized that equivalent sutures may be provided by other manufacturers and suppliers and the invention is not considered limited to the Ethibond® sutures used for purposes of disclosure. A traction suture 116 of ______ cm [Dr. Knowles? length has been found suitable for the application.

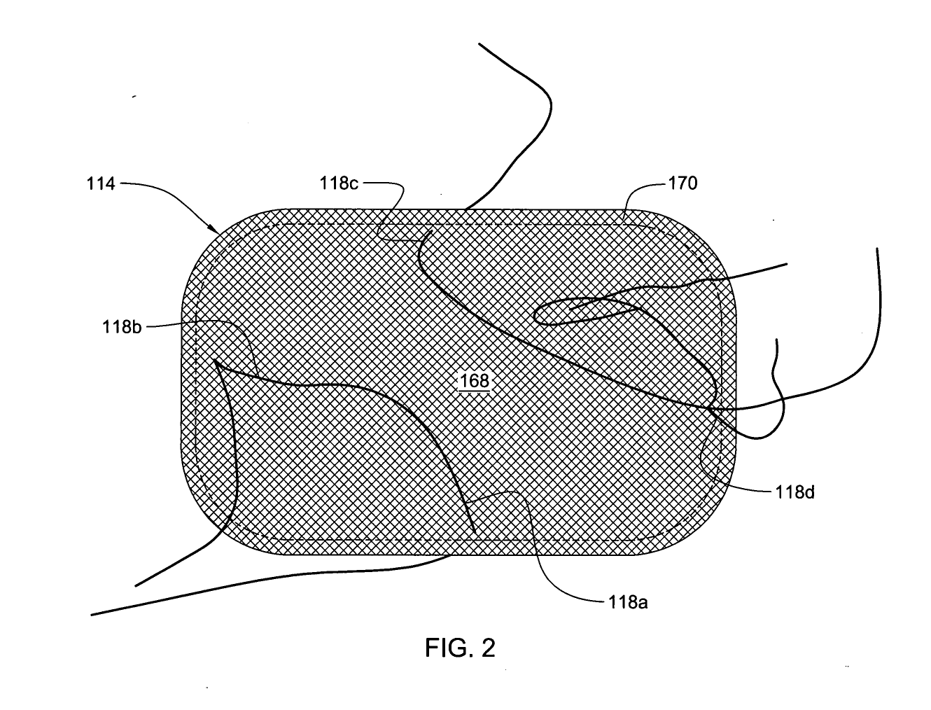

Referring now also to FIG. 2, there is shown a top, perspective, schematic view surgical mesh 114. Four traction or retention sutures 118a . . . 118d are attached peripherally adjacent the edge of surgical mesh 114. Sutures 118a . . . 118d are placed at approximately the midpoint of each edge, not specifically identified, of surgical mesh 114 (i.e., at approximately 12, 3, 6, and 9 o'clock positions).

Once insertion tube 102 has been suture ligated, and traction or retention sutures 118a . . . 118d have been placed in surgical mesh 114, surgical jelly from syringe 108 is injected into the open distal end 106 of insertion tube 102.

Next, a small nick 122 is made completely through the wall into the lumen of insertion tube 102 adjacent the traction suture 116 and between traction suture 116 and distal end 106 of insertion tube 102.

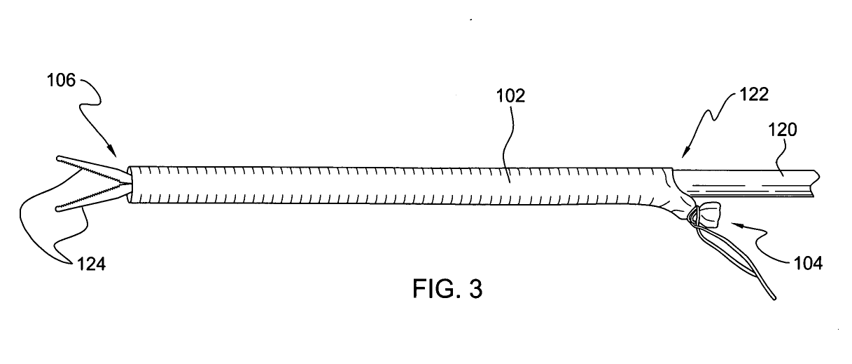

Referring now also to FIG. 3, a 5 mm laparoscopic grasper 120, for example, a Maryland grasper or dissector, is inserted into a hole formed in insertion tube 102 at nick 122. The shaft of laparoscopic grasper 120 is pushed through the previously lubricated lumen of insertion tube 102 until its grasping jaws 124 emerge from distal end 106 of inspection tube 102.

Surgical mesh 114 is next hydrated and rolled tightly into a cigar-like configuration. Traction sutures 118a . . . 118d are contained within the tightly rolled surgical mesh 114.

Next, the grasping jaws 124 or laparoscopic grasper 120 are manipulated so as to grab an edge of tightly rolled surgical mesh 114. Tightly rolled surgical mesh 114 is drawn into the lumen of insertion tube 102 as laparoscopic grasper 120 is withdrawn therefrom. Because insertion tube 102 is compliant, the combination of the hydrated surgical mesh 114 and the surgical jelly allows the rolled surgical mesh 114 to be readily slid into the lumen of insertion tube 102.

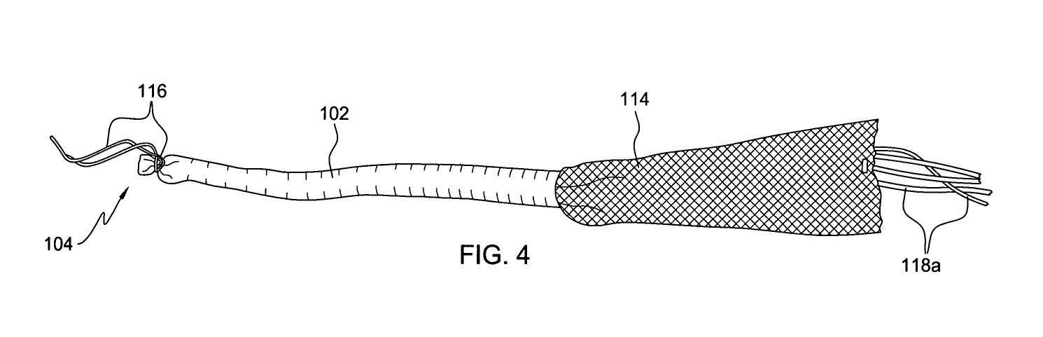

Referring now also to FIG. 4, insertion tube 102 is now loaded and ready for insertion in the abdominal cavity, not shown, of a patient, not shown. It is assumed that a laparoscope, not shown, is in proper position within the patient's abdominal cavity, that the abdominal cavity has been insufflated with carbon dioxide gas, and any other tasks normally associated with laparoscopic surgery have been accomplished. Such tasks are well known to practitioners of laparoscopic surgery and form no part of the method of the invention. Consequently, such tasks are not further described herein.

At least two 5-mm trocars have already been or are now inserted into the patient's abdomen at sites selected by the surgeon.

A second 5 mm laparoscopic grasper, not specifically identified, is passed into the abdominal cavity through a first of the at least two 5 mm trocars. The second 5 mm laparoscopic grasper is passed through the insufflated abdomen and back out through the second of the at least 5 mm trocars.

The second of the at least two 5 mm trocars is then removed, leaving at least the jaws of the second 5 mm grasper protruding from the patient's abdomen.

The grasping jaws of the second 5 mm grasper are then actuated to grasp the protruding the ligated proximal end 104 of insertion tube 102 and/or a portion of traction suture 116.

The second 5 mm grasper is then retracted thereby pulling the Traction suture 116 and, ultimately, the proximal end 104 of insertion tube 102 into the patient's abdominal cavity. Continued withdrawal of the second 5 mm grasper succeeds in pulling at least the major portion of insertion tube 102 containing the tightly rolled surgical mesh 114 completely into the abdominal cavity.

Insertion tube 102 is then parted along its longitudinal axis or otherwise opened to free the tightly rolled surgical mesh 114. Parting is accomplished using any combination of suitable instruments and surgical techniques not discussed herein.

Once tightly rolled surgical mesh 114 is free, insertion tube 102 is withdrawn from the abdominal cavity and the second 5 mm trocar is reinstalled in the incision originally made thereby.

Hernia repair or other surgery may then proceed using conventional techniques well known to practitioners of laparoscopic surgery.

The most difficult step of this novel process has been found to be the parting of insertion tube 102 and the freeing of tightly rolled surgical mesh 114 from the parted insertion tube 102. This has led to the development of another aspect of the present invention.

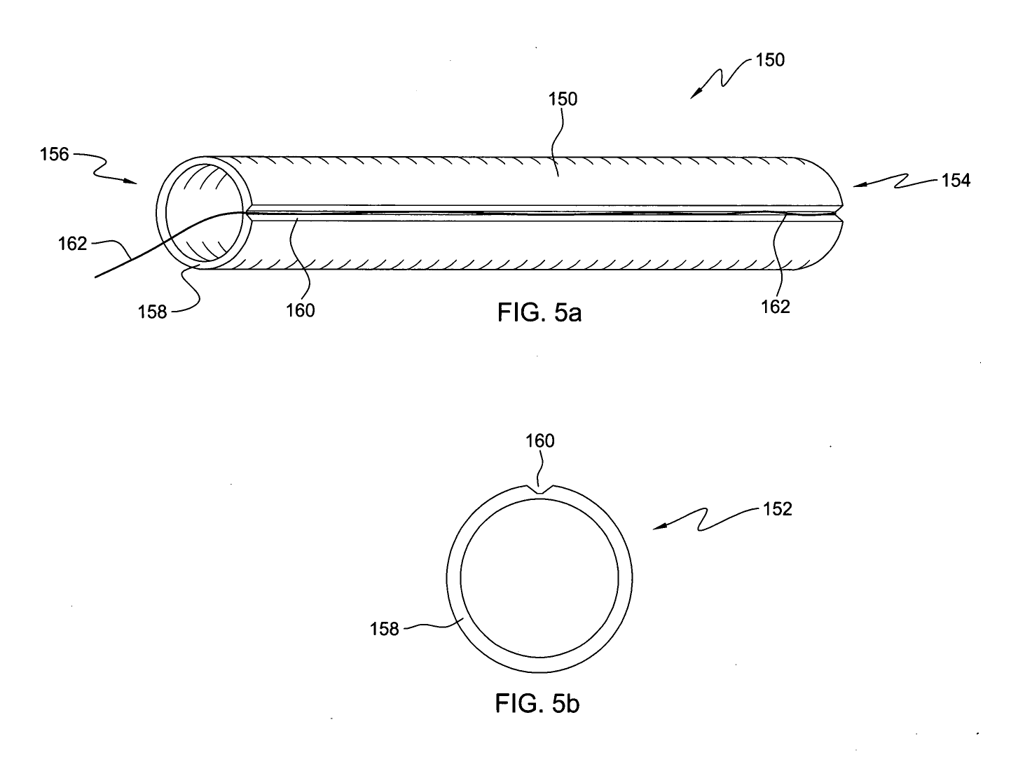

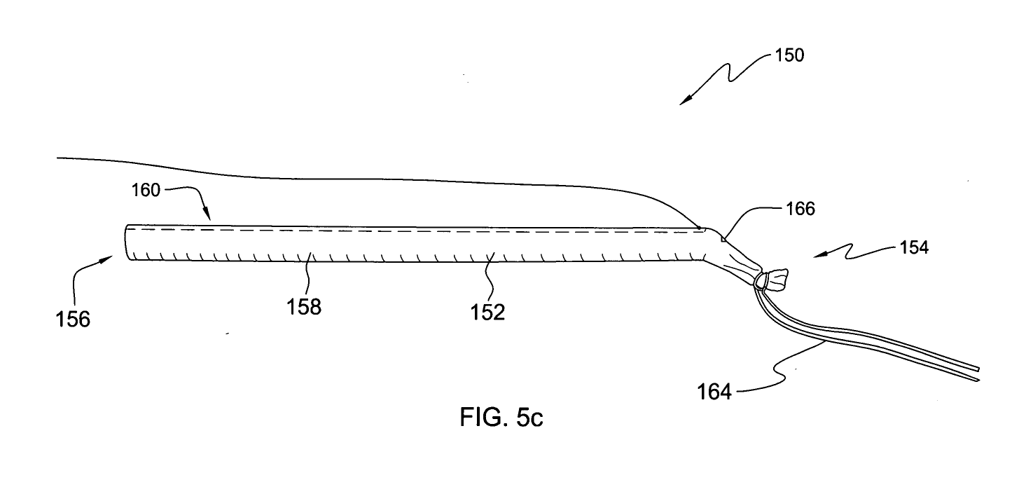

Referring now to FIGS. 5a, and 5b, there are shown side, perspective and end elevational schematic views, respectively, of an improved insertion tube 150. Insertion tube 150 features a substantially cylindrical, elongated body 152. Substantially cylindrical body 152 has a proximal end 154, a distal end 156, and a wall 158. Wall 158 has a thinned area 160 running along the major, longitudinal axis of body 152. Thinned area 160 may run along the entire length of body 152 or, in alternate embodiments, may run along only a portion of the length of body 152.

A ripcord 162 is attached to body 152 at thinned area 160 adjacent proximal end 154. Ripcord 162 may be passed through the lumen of body 152, extending beyond distal end 156 thereof. In alternate embodiments, ripcord 162 may be left outside body 152.

Referring now also to FIG. 5c, body 152 may be provided with a ligation using a traction suture 164 such as an Ethibond® or an equivalent suture, disposed adjacent proximal end 154. In alternate embodiments, a suitable ligating traction suture 164 may be installed in situ at the time of use of insertion tube 150.

Further, insertion tube 150 may be provided with an appropriate nick 166 adjacent traction suture 164 or, alternately, a nick 166 may be placed in insertion tube 150 at the time of use.

The improved insertion tube is used in a similar fashion to the insertion tube 102 discussed in detail hereinabove. However, at the step in the surgical process where the tightly rolled surgical mesh is to be freed from insertion tube 150, ripcord 162 is pulled from the distal end of body 152. The ripcord then separates (i.e., “unzips”) insertion tube 150 along weakened area 160, thereby freeing tightly rolled surgical mesh 114 from insertion tube 150. This eliminates the need for manipulation of laparoscopic instruments within the abdominal cavity and reduces the time required for the surgery.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A method of inserting surgical mesh into the body of a patient undergoing laparoscopic surgery, the steps comprising:
a) providing a substantially cylindrical, elongated insertion tube having an open proximal end and an open distal end;
b) ligating said insertion tube proximate said proximal end thereof;
c) forming a opening through a wall of said insertion tube adjacent said ligation at a position between said ligation and said distal end of said insertion tube;
d) preparing a piece of surgical mesh and rolling said surgical mesh into a tight roll;
e) passing a grasping instrument into a lumen of said insertion tube via said opening formed through said wall and advancing said instrument along said lumen of said insertion tube until a grasping end of said grasping instrument emerges from said insertion tube at said distal end thereof;
f) grasping said tightly rolled surgical mesh with said grasping instrument; and
g) pulling said tightly rolled surgical mesh into said lumen of said insertion tube.

2. The method of inserting surgical mesh into the body of a patient undergoing laparoscopic surgery as recited in claim 1, the steps further comprising:
h) prior to said passing a grasping instrument into a lumen of said insertion tube step (e), lubricating said lumen of said insertion tube.

3. The method of inserting surgical mesh into the body of a patient undergoing laparoscopic surgery as recited in claim 2, wherein said lubricating step (h) comprises injecting surgical jelly into said lumen.

4. The method of inserting surgical mesh into the body of a patient undergoing laparoscopic surgery as recited in claim 3, wherein said lubricating step (h) comprises injecting surgical jelly into said lumen using a syringe having a soft, tapered tip.

5. The method of inserting surgical mesh into the body of a patient undergoing laparoscopic surgery as recited in claim 1, wherein said preparing a piece of surgical mesh step (d) comprises the sub-step: prior to rolling said surgical mesh into a tight roll, hydrating said surgical mesh.

6. The method of inserting surgical mesh into the body of a patient undergoing laparoscopic surgery as recited in claim 1, wherein said preparing a piece of surgical mesh step (d) comprises the sub-step: prior to rolling said surgical mesh into a tight roll, placing at least one traction suture into said surgical mesh at a position along a perimeter thereof.

7. The method of inserting surgical mesh into the body of a patient undergoing laparoscopic surgery as recited in claim 6, wherein said placing at least one traction suture into said surgical mesh sub-step comprises placing four traction sutures into said surgical mesh at a position along a perimeter thereof.

8. The method of inserting surgical mesh into the body of a patient undergoing laparoscopic surgery as recited in claim 7, wherein said placing four traction sutures into said surgical mesh at a positions along a perimeter thereof sub-step comprises placing one of said four traction sutures at approximately a midpoint of each edge of said surgical mesh and adjacent a perimeter thereof.

9. The method of inserting surgical mesh into the body of a patient undergoing laparoscopic surgery as recited in claim 6, wherein said ligating step (b) comprising ligating said insertion tube using a braided, polymeric suture.

10. The method of inserting surgical mesh into the body of a patient undergoing laparoscopic surgery as recited in claim 9, wherein said preparing a piece of surgical mesh step (d) comprises the sub-step: prior to rolling said surgical mesh into a tight roll, placing at least one traction suture into said surgical mesh at a position along a perimeter thereof.

11. The method of inserting surgical mesh into the body of a patient undergoing laparoscopic surgery as recited in claim 10, wherein said grasping step (f) comprises grasping at least one selected from the group: said braided polymeric suture, and an edge of said surgical mesh.

12. The method of inserting surgical mesh into the body of a patient undergoing laparoscopic surgery as recited in claim 1, wherein said forming an opening step (c) comprises forming a nick in said wall of said insertion tube.

13. The method of inserting surgical mesh into the body of a patient undergoing laparoscopic surgery as recited in claim 1, the steps further comprising:
h) inserting a laparoscopic grasping instrument into the abdomen of said patient through a first 5 mm trocar;
i) passing said laparoscopic grasping instrument through said abdomen of said patient and back out through a second 5 mm trocar;
j) removing said second trocar after said laparoscopic grasping instrument emerges therefrom.

14. The method of inserting surgical mesh into the body of a patient undergoing laparoscopic surgery as recited in claim 13, the steps further comprising:
k) grasping at least one selected from the group: said braided polymeric suture, and said proximal end of said insertion tube with a grasping element of said laparoscopic grasping element; and
l) pulling at least a portion of said insertion tube carrying said tightly rolled surgical mesh into the abdomen of said patient;
m) releasing said tightly rolled surgical mesh from said insertion tube; and
n) withdrawing said insertion tube from the patient's abdomen.

15. The method of inserting surgical mesh into the body of a patient undergoing laparoscopic surgery as recited in claim 14, the steps further comprising:
o) replacing said second 5 mm trocar in its original incision in the abdomen of said patient.

16. An insertion tube for laparoscopically placing a surgical mesh in a body cavity of a patient, comprising:
a) an elongated, hollow, flexible tube having an open proximal end and an open distal end, a predetermined wall thickness, and a lumen formed therein;
b) a region disposed along the longitudinal axis of said hollow, elongated tube wherein said wall is thinned and a wall thickness measured at said region is less than said predetermined wall thickness; and
c) a ripcord operatively attached at a proximal end thereof to said elongated hollow tube proximate said proximal end at said thinned wall region;
whereby, when a force is applied to a distal end of said ripcord, said elongated hollow tube is split apart at said thinned wall region.

17. The insertion tube for laparoscopically placing a surgical mesh in a body cavity of a patient as recited in claim 16, wherein said ripcord is passed from said proximal end of said insertion to said distal end of said insertion tube and said distal end of said ripcord extends therebeyond.

18. The insertion tube for laparoscopically placing a surgical mesh in a body cavity of a patient as recited in claim 17, wherein said ripcord is passed from said proximal end of said insertion to said distal end of said insertion tube within said lumen of said insertion tube.

19. The insertion tube for laparoscopically placing a surgical mesh in a body cavity of a patient as recited in claim 16, further comprising:
d) a ligation adjacent said open proximal end.

20. The insertion tube for laparoscopically placing a surgical mesh in a body cavity of a patient as recited in claim 19, further comprising:
e) a nick through a wall of said insertion tube adjacent said ligation at a position between said ligation and said distal end of said insertion tube.
